(19) United States
(12) Patent Application Publication
(10) Pub. No.: US 2021/0113366 A1
(43) Pub. Date: Apr. 22, 2021

(54) Thermal Therapy Garment System

(71) Applicant: Brandon Roberts, Centerville, OH (US)

(72) Inventor: Brandon Roberts, Centerville, OH (US)

(21) Appl. No.: 15/929,785

(22) Filed: May 21, 2020

Related U.S. Application Data
(63) Continuation-in-part of application No. 15/807,533, filed on Nov. 8, 2017, now abandoned.
(60) Provisional application No. 62/528,265, filed on Jul. 3, 2017.

(51) Int. Cl. A61F 7/02; A41D 13/00; A61F 13/08; A41D 1/04; A41B 1/08; A41D 1/089; A41D 1/02

(52) U.S. Cl. CPC A61F 7/02; A41D 13/0015; A61F 13/08; A61F 2007/0009; A41B 1/08; A41D 1/089; A41D 1/02; A41D 1/04

(57) ABSTRACT

A thermal therapy garment system includes a garment assembly having a compression garment including an outer surface, an inner surface having a pocket, and a thermal pack. The pocket is located on the inner surface of the compression garment and is strategically placed on the compression garment such that thermal therapy is enabled on a target area of a body of a user-wearer. The target area of the body of the user-wearer is a muscle, a joint, or both. A compression garment may be worn underneath everyday clothing and may be concealed under everyday clothing.

### CROSS-REFERENCE TO RELATED APPLICATION

The present application is a continuation-in-part of U.S. Non-Provisional patent application Ser. No. 15/807,533, filed Nov. 8, 2017, pending U.S. Provisional Patent Application Ser. No. 62/528,265 filed Jul. 3, 2017, expired, both of which are incorporated by reference herein in their entirety.

### BACKGROUND OF THE INVENTION

The following includes information that may be useful in understanding the present disclosure. It is not an admission that any of this information is prior art nor is material to the present therapy device, nor that any explicitly or implicitly referenced publication or document is prior art.

### FIELD OF THE INVENTION

The present invention relates to the field of therapy devices.

### DESCRIPTION OF RELATED ART

Many people use ice or heat packs on injuries. An ice pack is a portable object filled with a cold liquid. Placement over an injured area absorbs heat from an injury. A heat pack is a warm portable object to supply heat to an injury. Both can alleviate minor injury pains and decrease muscle soreness. Ice- or heat-pack therapy typically requires individuals to rest or hold the packs on their bodies in an awkward position. Ice can begin to drip and leak over clothing and furniture, creating a huge mess. People may start to get frustrated, dealing with applying ice-and heat-packs to the back, neck, legs, etc. Having to continually apply ice or heat can impact a person's everyday life, leaving them unable to enjoy physical activities such as jogging, cycling, hiking, and more. A suitable solution is desired.

U.S. Pub. No. 2008/0125842 to Steve Petitt relates to a thermal, elastic, tight-fitting garment with pockets positioned for thermal therapy. The described thermal, elastic, tight-fitting garment with pockets positioned for thermal therapy includes a form-fitting elastic garment having a pocket corresponding to a group of muscles or tendons. The pockets are adapted to receive a thermal pack and hold the thermal pack tightly against a group of muscles or tendons. A thermal therapy kit includes a thermally insulated container storing a thermal medium and such a form-fitting garment.

### BRIEF SUMMARY OF THE INVENTION

The present disclosure provides a novel thermal therapy garment system. The general purpose of the present disclosure is to provide form-fitting garments, including a partial turtleneck, t-shirt, compression pants, long socks, and short socks with built-in compartments stitched into the garment for fitting ice and heat packs.

The system includes garment assembly having a compression garment including an outer surface, and an inner surface having a pocket, and a thermal pack configured to provide thermal therapy to a user-wearer.

The garment assembly includes a compression garment and a thermal pack. These thermal packs have various sizes, fit within pockets in the garment assembly, and sit on the inner surface of the compression garment. The pockets occupy strategic positions on the compression garment over a target area of a user's body, including, among other positions, positions over muscles, muscle groups, and joints. A compression garment may include a long-sleeved shirt, a short-sleeved shirt, pants, shorts, ankle socks, hi-compression socks, a vest, a shoulder and neck wrap, a sweater, or a jacket or combination thereof. The compression garment holds the thermal pack tightly against the target area of the body of the user. The target area of the body of the user-wearer is a muscle, a joint, or both. The inner surface touches the user's body as thermal therapy happens.

This disclosure concerns certain aspects, advantages, and novel features of the thermal therapy system, as set out below. Not necessarily all such advantages may be achieved following any one particular embodiment of the invention. Thus, the manner of carrying out the system may achiever optimize one advantage or group of advantages without necessarily achieving other disclosed advantages.

### BRIEF DESCRIPTION OF THE DRAWINGS

The figures that accompany the written portion of this specification illustrate embodiments and methods of use for a thermal therapy garment system, constructed and operative according to this disclosure.

FIG. 1A is a perspective view of a component of the thermal therapy garment system during an ‘in-use’ condition, according to an embodiment of the disclosure.

FIG. 1B is a perspective view of the FIG. 1A component.

FIG. 2A is a perspective view of another system component.

FIG. 2B is a back perspective view of the FIG. 2A component.

FIG. 3A is a front view of another system component.

FIG. 3B is a back view of the FIG. 3A component

FIG. 4A is a front view of another system component.

FIG. 4B is a back view of the FIG. 4A component

FIG. 5A is a side view of another system component.

FIG. 5B is a front perspective view of the FIG. 5A component.

FIG. 6A is a front view of a pattern piece of a system component.

FIG. 6B is a back view of a pattern piece of the FIG. 6A component.

FIG. 6C is an upper view of a left-side pattern piece of the FIG. 6A component.

FIG. 6D is an upper view of a right-side pattern piece of the FIG. 6A component.

FIG. 7A is a front view of a pattern piece of another system component.

FIG. 7B is a back view of a pattern piece of the FIG. 7A component.

FIG. 7C is an upper view of a right-side pattern piece of the FIG. 7A component.

FIG. 7D is an upper view of a left-side pattern piece of the FIG. 7A component.

FIG. 8A is a perspective view of a system component.

FIG. 8B is a front view of the FIG. 8A component.

FIG. 8C is an upper view of a pattern piece of the FIG. 8A component.

FIG. 9A is a front view of a pattern piece of another system component.

FIG. 9B is a back view of a pattern piece of the FIG. 9A component.

FIG. 9C is a front view of another system component.

FIG. 9D is a side view of another system component.

FIG. 10A is a front view of another system component.

FIG. 10B is a back view of the FIG. 10A component.

FIG. 10C is a side view of the FIG. 10A component.

FIG. 11A is a perspective view of another system component.

FIG. 11B is a perspective view of another system component.

FIG. 11C is a perspective view of another system component.

The various embodiments of the present invention with the appended drawings disclosed the current therapy system.

### DETAILED DESCRIPTION

As discussed above, embodiments of the present disclosure relate to a therapy device: a thermal therapy garment system used to improve thermal therapy.

Generally, the present invention provides users with compression garments equipped with small pockets designed to removably receive ice or heat packs for pain relief. It includes multiple compartments stitched (or otherwise fastened) into each garment, allowing wearers to target specific areas of the body for applying ice or heat packs. These compartments may mount on the inner surface of the garment. This mounting provides concealment that allows individuals to wear the garments and packs underneath their everyday clothing for extended door all-day relief, enabling people to maintain a functional lifestyle. The thermal system uses five different garments: a neck-and-shoulder garment (such as a hoodie), a shirt, pants, and high compression and ankle socks, offering full-body relief for back pain, neck, or shoulder pain, upper and lower leg pain, and foot pain. A user may insert ice or heat packs into the pockets from the inside of the garment and may remove the ice or heat packs once done. But the user may continue with regular activity, wearing the garment after therapy. The inside pockets or inner pockets are not visible, and a user may wear the garment without ice or heat packs. The garment substantially matches the appearance of regular compression wear. Other garment-types may be available.

The present invention effectively eliminates the need to hold an ice or heat pack on the body or to use uncomfortable and hindering accessories to obtain hot or cold relief from aches and pains. The thermal therapy garment system is a set of modified compression garments integrated with multiple pockets or compartments designed to hold ice or heat packs. The garments can comprise a partial turtleneck, a compression shirt, pants, or high-compression or low ankle socks. Each garment may include rectangular or square pockets directly sewn into the garment; the ankle socks can feature a circular compartment capable of receiving a round ice pack.

A compression shirt can be worn underneath a standard shirt and allows individuals to target core and back muscles for ice/heat cooling/heating relief. The pants can be worn underneath shorts, pants, or other garments and can apply ice and heat to upper-leg and shin areas. The high socks can apply therapy to injured shin or calf areas. Further, the ankle socks can apply ice or heat to an injured ankle area. Various garment versions comprise any one or any combination of polyester, cotton, spandex, elastin, and other suitable materials or fabrics. The garments may be available in numerous sizes to accommodate all user-wearer needs and preferences. Exact size, measurement, construction, and design specifications may vary upon manufacturing.

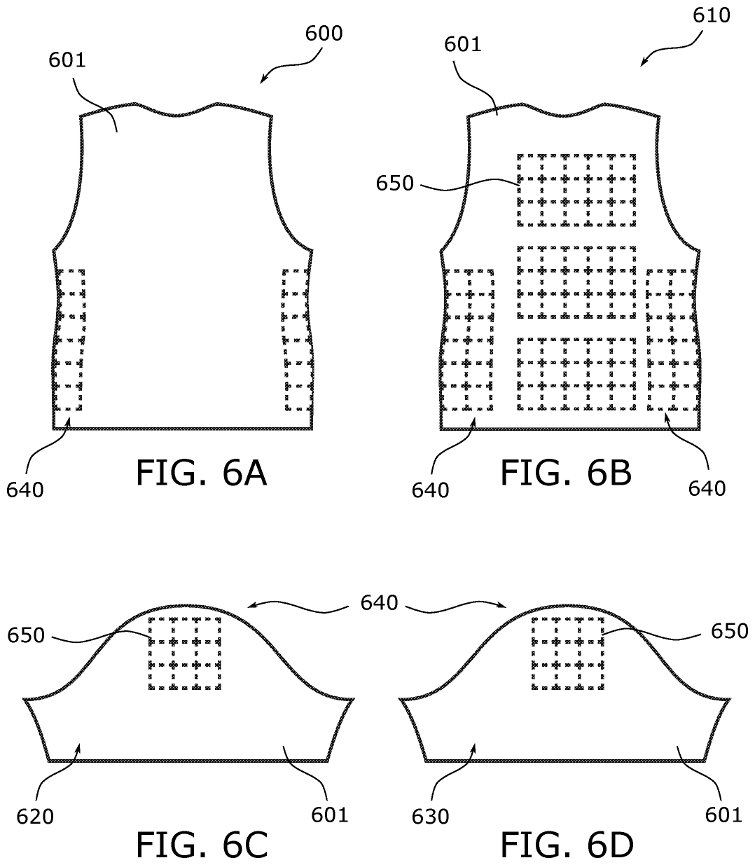

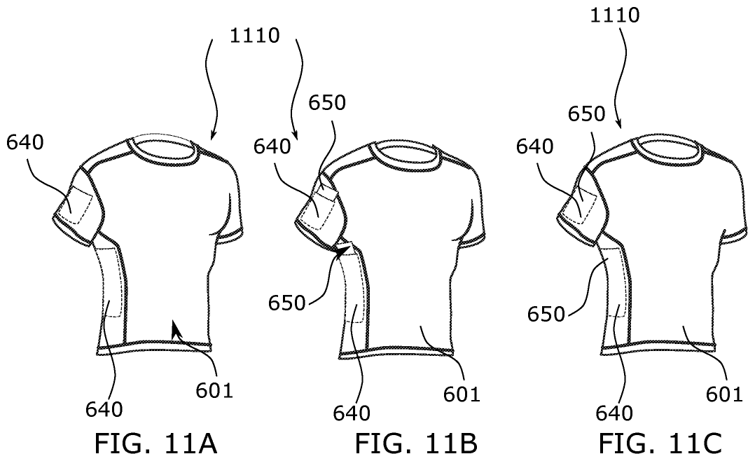

FIGS. 6A through 11C show components of a thermal therapy system 100. FIG. 6A depicts front shirt panel 600 constructed out of any desirable fabric, as is known to those of skill in the art. The figure also shows inner pocket 130. Inner pocket 130 firmly and securely holds a thermal pack (FIG. 11B, thermal pack 1120). Inner pocket 130 is reinforced and attached to the inside of the garment using stitching 650. Inner pocket 130 is shown along the upper body below the arms of a user. Depending on the embodiment, inner pocket 130 sits anywhere that thermal treatment would be considered useful. FIG. 6B shows a back shirt panel 610 with stitching 650 connecting inner pocket 130 with front shirt panel 600 and strengthens pocket 130. The garment of the system version shown in FIG. 6B has several inner pockets 130 on back shirt panel 610.

Some of the pockets 130 sit close to the user's back. This arrangement provides close contact between a user's body and thermal pack 1120 (in pocket 130). The figure also depicts inner pocket 130 wrapping around the user's side to the back, continuations of the inner pockets 130 from the user's sides shown in FIG. 6A. In some versions, “pocket” means a pouchlike structure formed by connecting a smaller component to a larger component onto or three sides of the smaller component. In some versions, the garment does not have a slit in an outer layer of fabric. In some versions, the compression garment has a single layer of compression fabric

FIGS. 6C and 6D represent left shirtsleeve panel 620 and right shirtsleeve panel 630. In these figures, panel 620 and 630 are flattened to illustrate details of the garment. Panel 620 and panel 630 also carry inner pocket 130. Stitching 650 attaches inner pocket 130 to panel 620 and 630 and strengthens inner pocket 130. In this version of the garment system, installing thermal pack 1120 in inner pocket 130 holds thermal pack 1120 close to the region transitioning from the user's shoulder to their upper arm.

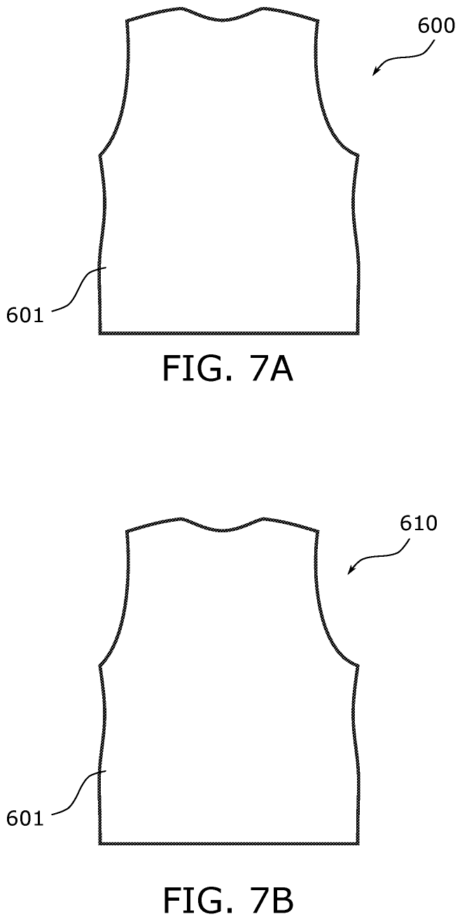

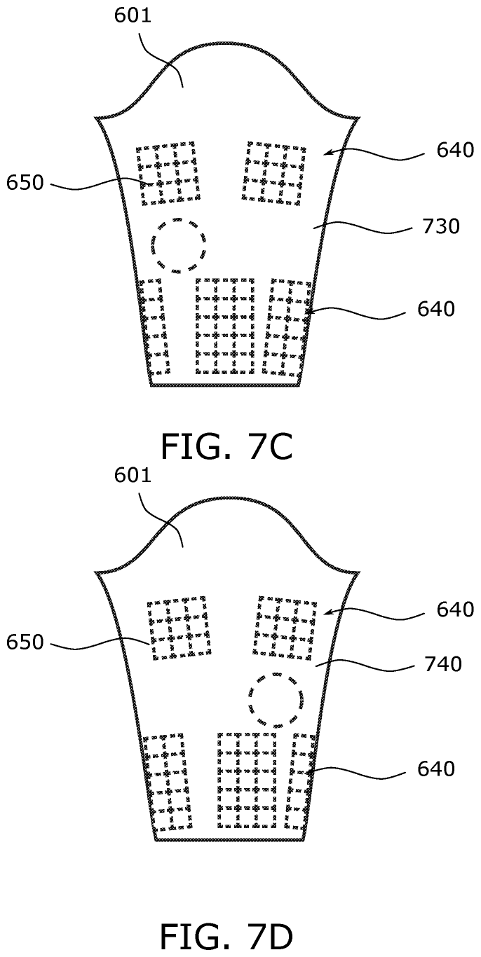

FIG. 7A depicts front shirt panel 600 for a different version of a thermal therapy garment. Likewise, FIG. 7B depicts back shirt panel 610 of the garment depicted in FIG. 7A. This version has long sleeves, as shown in FIG. 7C and FIG. 7D. The figures depict left long-sleeve panel 730 and right sleeve panel 740. As with the short-sleeve version, long-sleeve panel 730 and long-sleeve panel 740 are shown flattened to illustrate details of the garment system. As with the other components, long-sleeve panel 730 and long-sleeve panel 740 carry inner pockets 130. And stitching 650 connects inner pockets 130 to panel 730 and 740 and strengthens inner pocket 130. Installation of thermal pack 1120 into pocket 130, holds thermal pack 1120 against a user's forearm and upper arm.

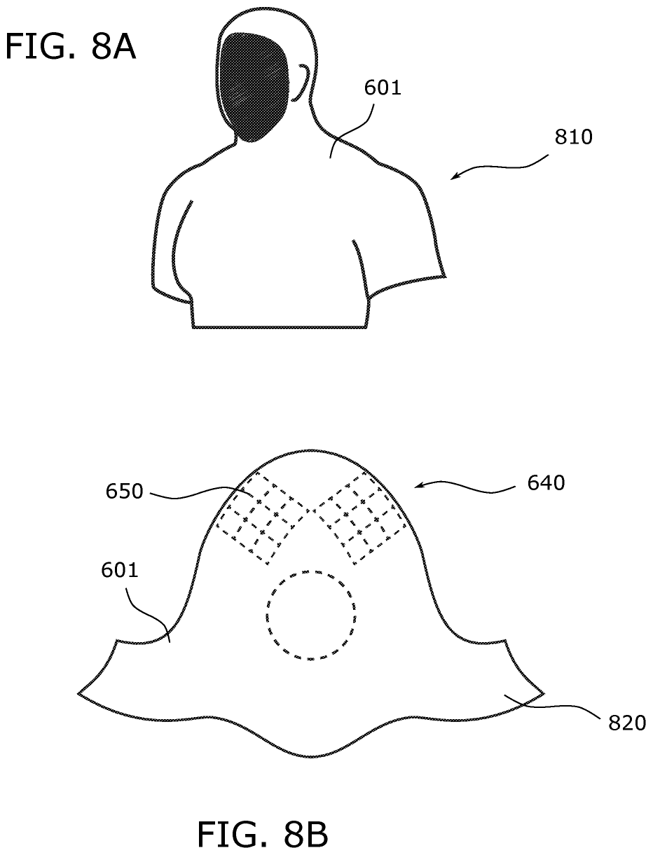

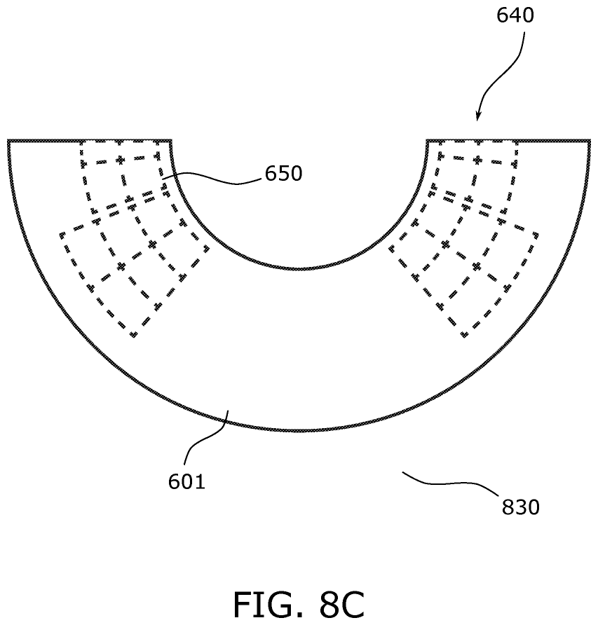

FIG. 8A depicts a hoodie 810 version of a thermal therapy garment. FIG. 8B depicts hood panel 820 separated from hoodie 810 and flattened, as were the sleeves, above. Hood panel 820 carries inner pocket 130. As before, stitching 650 connects inner pocket 130 to hood panel 820 and strengthens inner pocket 130.

FIG. 8C depicts neck panel 830 from hoodie 810. Hoodie 810 also carries inner pocket 130, and stitching 650 also connects inner pocket 130 to hoodie 810 and strengthens inner pocket 130.

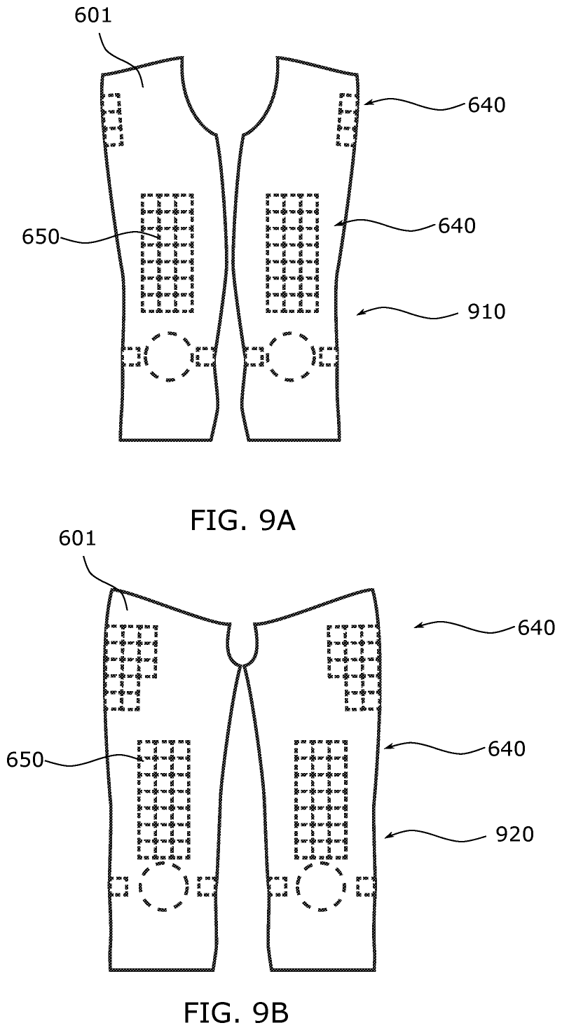

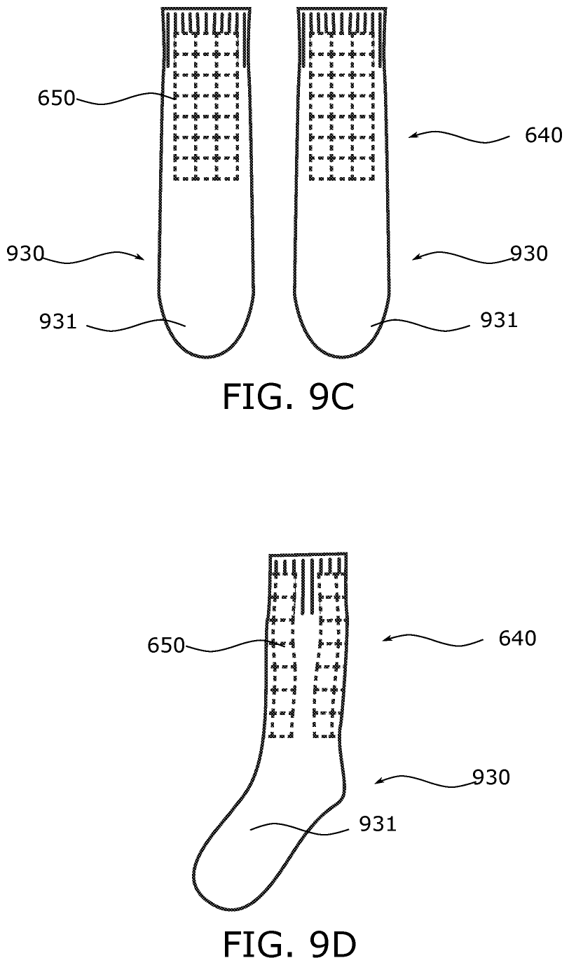

FIG. 9A depicts a pants version of the therapy garment. Front pants panel 910 carry inner pockets 130. Stitching 650 connects inner pocket 130 to front pants panel 910 and strengthens inner pocket 130. FIG. 9B is substantially the same except it depicts back pants panel 920. In the particular versions disclosed in FIGS. 9A and 9B, inner pocket 130 sits such that the installation of thermal pack 1120 in inner pocket 130 provides treatment to a user's hips and legs (front and back sides).

FIGS. 9C and 9D depict two sides 930 that have inner pockets 130 sewn on and strengthen with stitching 650. Sock 930 comprises any fabric material 931 that is known to be useful for socks. The version depicted in FIGS. 9C and 9D have inner pocket 130 sitting such that, when thermal pack 1120 is installed in inner pocket 130, sides 930 supply thermal therapy to a user's lower calves.

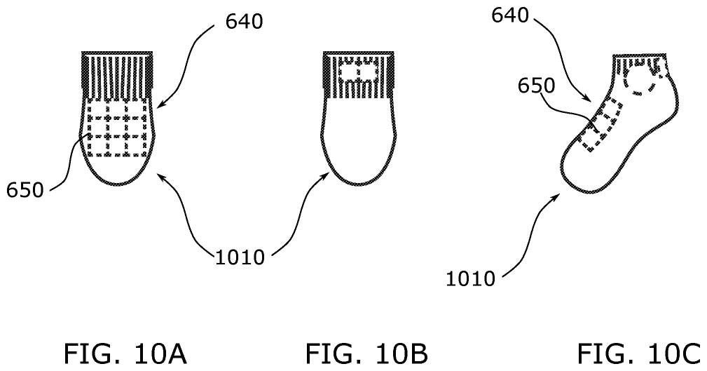

FIGS. 10A through 10C depict short socks 1010 having inner pocket 130 sewn on and strengthened with stitching 650. In this version of the garment system, inner pocket 130 sits on the inside of sock 1010, such that the installation of thermal pack 1120 in inner pocket 130 applies thermal therapy to the top of the user's foot.

FIGS. 11A through 11C depict a T-shirt version 1110 of a thermal garment system. FIG. 11A shows T-shirt version 1110 with examples of inner pocket 130 on the top of the sleeve for thermal therapy on a user's upper arm and inner pocket 130 located below the arm to provide therapy along the sides of the user's chest. FIG. 11B shows the same T-shirt as in FIG. 11A. This figure shows thermal pack 1120 partially inserted into inner pockets 130. Both inner pocket 130 and thermal pack 1120 are inside the fabric of T-shirt 1104, in close contact with the user. FIG. 11C shows the T-shirt depicted in FIGS. 11A and 11B. This figure shows both thermal pack 1120 fully inserted into inner pocket 130.

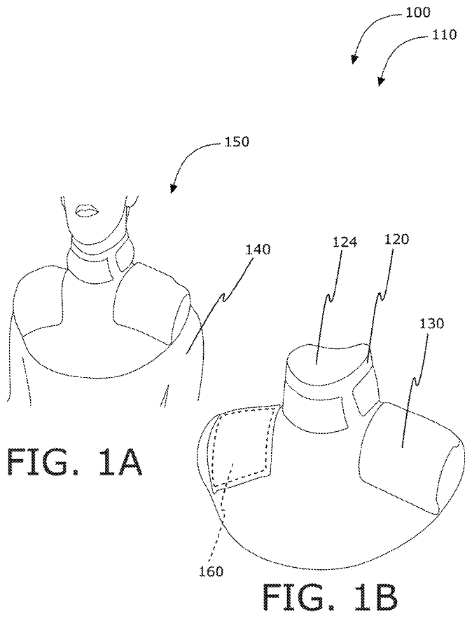

FIGS. 1A-5B show various views of a thermal therapy garment system 100. FIG. 1A shows thermal therapy garment system 100 during an ‘in-use’ condition. As illustrated, system 100 may include garment assembly 110 having a compression garment 120, including an outer surface 128, an inner surface 124, inner pocket 130, and thermal pack 160. Compression garment 120 may include a long-sleeved shirt, a short-sleeved shirt, pants, shorts, ankle socks, hi-compression socks, a vest, a shoulder-and-neck wrap, a sweater, or a jacket. Versions having different arrangements of these garment types are also contemplated.

Garment assembly 110 comprises compression garment 120 and thermal pack 160; thermal packs 160 fit within inner pockets 130. Inner pockets 130 sit on inner surface 124. Inner pockets 130 are strategically placed on compression garment 120 to provide thermal therapy to a target area. Compression garment 120 holds thermal pack 160 tightly against a target area such as a muscle, a joint, or both. And inner surface 124 is adjacent to the user pushing thermal pack 160 against the user.

In certain embodiments, a fastener may removably couple inner pocket 130 to compression garment 120. Hook-and-loop fasteners, snap fasteners, and zipper fasteners are all suitable fasteners. Compression garment 120 comprises a shoulder-and-neck wrap having inner pocket 130 disposed at a user's target body area. Compression garment 120 comprises a detachable hood having inner pocket 130 configured to receive the thermal pack 160. Inner pocket 130 comprises belt loops configured for securing thermal pack 160. Various versions of the therapy system contemplate using belt loops to secure thermal packs inside the inner pocket by means, such as by looping around thermal pack 160 in one or two directions.

Thermal pack 160 comprises a heat-pack or an ice-pack. Thermal pack 160 may be chosen based on user preference and therapy needs. Thermal pack 160 may be used selectively and in combination. Inner pocket 130 comprises, conceals, and holds thermal pack 160 tightly against the body target area. Compression garment 120 fits underneath street clothing. In some versions, compression garment 120 fits underneath street clothing so that users can conceal this pain-relief device. For purposes of this disclosure, “street clothing” includes clothing worn out of a person's home. In some versions, compression garment 120 is worn as underwear or undergarments. In some versions, compression garment 120 is worn over underwear or undergarments.

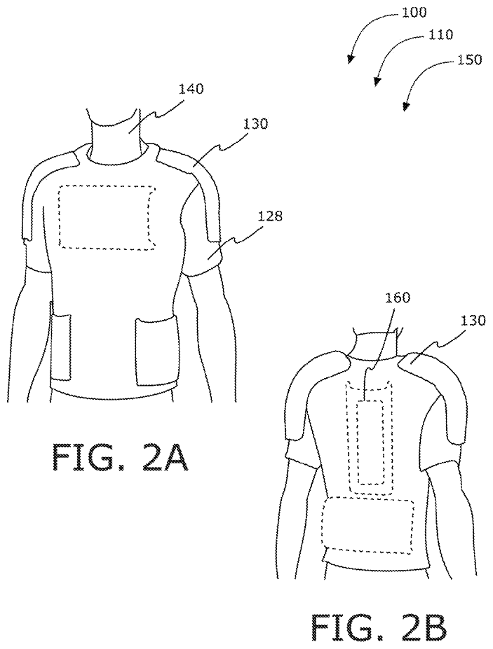

FIGS. 2A-2B depict thermal therapy garment system 100. As above, system 100 may include garment assembly 110 having compression garment 120, including outer surface 128, inner surface 124, and inner pocket 130. Compression garment 120 may include a short-sleeved shirt, as shown in FIGS. 2A-2B, having inner pocket 130 disposed at a target area including shoulders, chest, upper-back, lower-back, and sides. Compression garment 120 may further comprise a long-sleeved shirt having inner pocket 130 positioned to target the user's biceps, triceps, elbows, anterior forearms, posterior forearms, wrists, or combinations of these body parts.

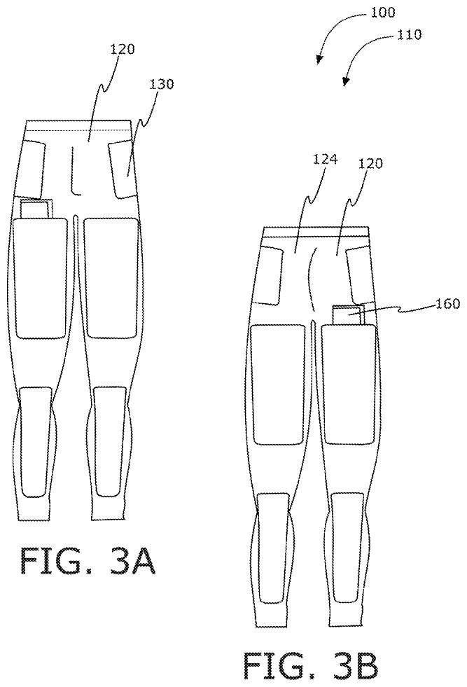

FIGS. 3A-3B show perspective views of system 100. As above, system 100 may include garment assembly 110 having compression garment 120, including outer surface 128 and inner surface 124, having inner pocket 130, and thermal pack 160. Compression garment 120 may include pants having inner pocket 130 positioned at a target area, including a hip, a quadricep, a hamstring, a shin, and a calf. A heat-pack or an ice-pack may be inserted selectively within inner pocket 130 to provide pain relief to the desired target area of the body.

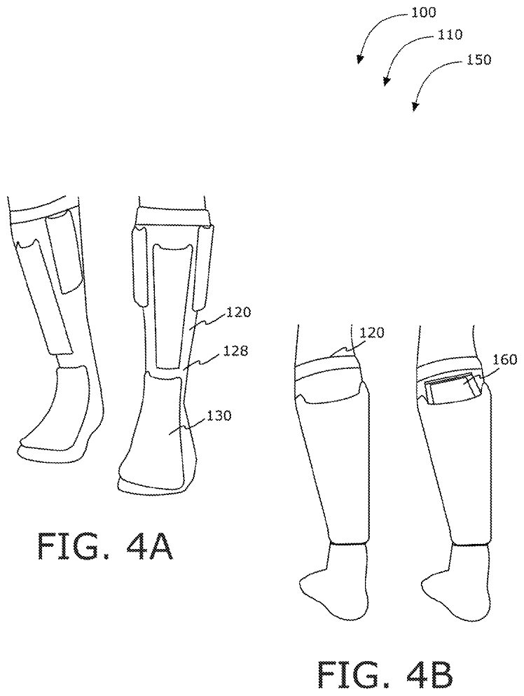

FIGS. 4A-4B show perspective views of the thermal therapy garment system 100. As above, system 100 may include garment assembly 110 having compression garment 120 having inner pocket 130 configured to receive thermal pack 160. Compression garment 120 may include hi-compression socks having inner pocket 130 disposed at a target area, including a shin, a calf, an ankle, and a foot. Different versions of system 100 comprise different inner-pocket 130 sizes depending on the size of the treatment area; inner pocket 130 may be larger to treat larger muscles and joints.

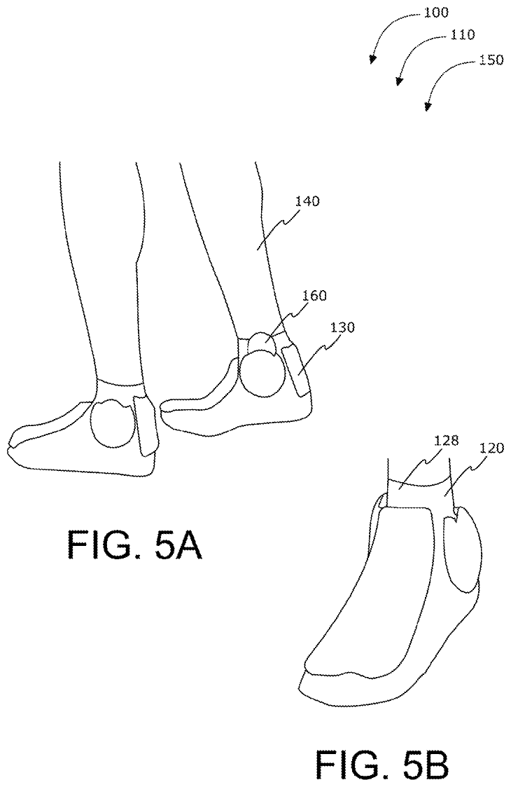

FIGS. 5A-5B show perspective views of system 100. As above, system 100 may include compression garment 120 having inner pocket 130 and thermal pack 160. Compression garment 120, as shown in FIGS. 5A-5B, may include ankle socks having inner pocket 130 disposed at a target area, including an ankle and a foot area. Inner pocket 130 may have a circular profile and may be disposed on a side or on opposing sides of an ankle. In some versions, inner pockets 130 may be square or rectangular or may sit on a front, back, or front and back side of the foot.

In some embodiments, the partial turtleneck fits under a standard shirt, and it contains 4-8 thick elastic belt loops that can hold a customized, flexible ice or heat pack in place to treat an injured neck or trapezius muscle. The inside compartment of the turtleneck can contain the belt loops to hold the pack in place. In a detachable hood version, with a shirt and partial turtleneck, the hood may attach using hook-and-loop (VELCRO) or zipper fasteners and may contain ice or heat compartments that can help people cool down from workouts or mitigate migraines or headaches.

The described embodiments are exemplary, and numerous modifications, variations, and rearrangements can substantially achieve equivalent results. All of these variations are within the spirit and scope of the invention.

What is claimed is:

1. A thermal therapy garment system comprising a garment assembly having:
a compression garment for a user including a component having
fabric with an outer surface and an inner surface, and
a pocket attached to the inner surface, and
a thermal pack,
wherein the pocket is located on the inner surface at a position corresponding to a target area of the user,
wherein the pocket holds the thermal pack tightly against the target area,
wherein the garment conceals the thermal pack, and
wherein the inner surface is adjacent to the user.

2. The system of claim 1, wherein the target area comprises a muscle.

3. The system of claim 2, wherein the target area further comprises a joint.

4. The system of claim 1, wherein the target area comprises a joint.

5. The system of claim 4, wherein the compression garment is any one of a long-sleeved shirt, a short-sleeved shirt, pants, shorts, ankle socks, hi-compression socks, a vest, a shoulder-and-neck wrap, a sweater, and a jacket.

6. The system of claim 5 further comprising one or more additional compression garments selected from any one or any combination of a long-sleeve shirt, short-sleeve shirt, pants, shorts, ankle socks, height-compression socks, a vest, shoulder-and-neck wrap, sweater, and a jacket.

7. The system of claim 1, wherein
the component comprises a shoulder-and-neck wrap and
the target area includes neck and trapezius muscles.

8. The system of claim 1 wherein the component is a detachable hood.

9. The system of claim 1, wherein the component comprises a long-sleeved shirt sleeve, and the target area is any one or any combination of biceps, triceps, elbows, anterior forearms, posterior forearms, and wrists.

10. The system of claim 9, wherein the target area includes biceps, triceps, elbows, anterior forearms, posterior forearms, and wrists.

11. The system of claim 1, wherein the component comprises a section of pants, and the target area comprises any one or any combination of a hip, a quadricep, a hamstring, a shin, or a calf.

12. The system of claim 11, wherein the target area includes a hip, a quadricep, a hamstring, shin, and a calf.

13. The system of claim 1, wherein the garment is an ankle sock, and the target area comprises a region of an ankle, a foot, or both.

14. The system of claim 1, wherein the garment is a hi-compression sock, and the target area comprises any one or any combination of a shin, a calf, an ankle, or a foot.

15. The system of claim 14, wherein the target area is a shin, a calf, an ankle, and a foot.

16. The system of claim 1, wherein the component comprises a short-sleeved shirt sleeve, and the target area is any one or any combination of shoulders, chest, and back.

17. The system of claim 16, wherein the target area includes shoulders, chest, and back.

18. The system of claim 1, wherein the thermal pack comprises a heat pack.

19. The system of claim 1, wherein the thermal pack comprises an ice pack.

20. The system of claim 1, wherein the compression garment fits underneath street clothing.
